SEQUENCE LISTING (7 sequences)

SEQ ID NO: 1 - protein, 226 aa, Artificial Sequence [sequence omitted]

SEQ ID NO: 2 - DNA, 684 bp, Artificial Sequence
  Synthetic Polynucleotide
aagacccacacctgtcctccctgtcccgcccctgaactgctgggaggccctagcgtgttcctgttccccccaaagcccaaggacaccctgatgatcagcaggacccccgaagtgacctgcgtggtggtggatgtgtctcacgaggaccctgaagtgaagttcaattggtacgtggacggcgtggaagtgcacaacgccaagaccaagcccagagaggaacagtacaacagcacctacagggtggtgtccgtgctgaccgtgctgcaccaggattggctgaacggcaaagagtacaagtgcaaggtgtccaacaaggccctgcctgcccccatcgaaaagaccatcagcaaggccaagggccagcccagggaaccccaggtgtacacactgccccccagcagggatgagctgaccaagaaccaggtgtccctgacctgtctcgtgaagggcttctacccctccgatatcgccgtggaatgggagagcaacggccagcccgagaacaactacaagaccaccccccctgtgctggacagcgacggctcattcttcctgtacagcaagctgacagtggacaagagcagatggcagcagggcaacgtgttcagctgcagcgtgatgcacgaggccctgcacaaccactacacccagaagtccctgagcctgagccccggcaaataatga

SEQ ID NO: 3 - protein, 361 aa, Artificial Sequence
  Synthetic Polypeptide
MDYYRKHAAVILATLSVFLHILHSFPDGEFTTQDCPECKLRENKYFFKLGVPIYQCKGCCFSRAYPTPARSRKTMLVPKNITSESTCCVAKAFIRVTVMGNIKLENHTQCYCSTCYHHKIGGGGSGGGGSGGGGSKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 1089 bp, Artificial Sequence
  Synthetic Polynucleotide
atggactactacagaaagcacgccgccgtgatcctggctaccctgtccgtgtttctgcacatcctgcacagcttccccgacggcgagttcacaacccaggactgccctgagtgcaagctgagagagaacaagtacttcttcaagctgggcgtgcccatctaccagtgcaagggctgctgcttcagcagggcctaccctacccccgccagatccagaaagaccatgctggtgcccaagaacatcaccagcgagagcacctgttgcgtggccaaggccttcatcagatggaccgtgatgggcaacatcaagctggaaaaccacacccagtgctactgctctacctgctaccaccacaagatcggcggaggcggaagtggcggcggaggatctgggggaggcggatctaagacccacacctgtcctccctgtcccgcccctgaactgctgggaggccctagcgtgttcctgttccccccaaagcccaaggacaccctgatgatcagcaggacccccgaagtgacctgcgtggtggtggatgtgtctcacgaggaccctgaagtgaagttcaattggtacgtggacggcgtggaagtgcacaacgccaagaccaagcccagagaggaacagtacaacagcacctacagggtggtgtccgtgctgaccgtgctgcaccaggattggctgaacggcaaagagtacaagtgcaaggtgtccaacaaggccctgcctgcccccatcgaaaagaccatcagcaaggccaagggccagcccagggaaccccaggtgtacacactgccccccagcagggatgagctgaccaagaaccaggtgtccctgacctgtctcgtgaagggcttctacccctccgatatcgccgtggaatgggagagcaacggccagcccgagaacaactacaagaccaccccccctgtgctggacagcgacggctcattcttcctgtacagcaagctgacagtggacaagagcagatggcagcagggcaacgtgttcagctgcagcgtgatgcacgaggccctgcacaaccactacacccagaagtccctgagcctgagccccggcaaataatga

SEQ ID NO: 5 - protein, 410 aa, Artificial Sequence
  Synthetic Polypeptide
METLQGLLLWMLLSVGGVWASRGPLRPLCRPINATLAAEKEACPICITFTTSICAGYCPSMVRVMPAALPAIPQPVCTYRELRFASIRLPGCPPGVDPMVSFPVALSCHCGPCQIKTTDCGVFRDQPLACAPQASSSSKDPPSQPLTSTSTPTPGASRRSSHPLPIKTSGGGGSGGGGSGGGGSKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - DNA, 1232 bp, Artificial Sequence
  Synthetic Polynucleotide
atggaaacactgcagggcctgctgctgtggatgctgctgtctgtgggcggagtgtgggccagcagaggacctctgaggcccctgtgcagacccatcaatgccacactggccgccgagaaagaggcctgccctatctgcatcaccttcaccaccagcatctgcgccggctactgcccttctatggtgcgcgtgatgcctgccgccctgcctgctattcctcagcccgtgtgcacctacagagagctgagattcgccagcatcaggctgcccggatgtcctcctggcgtggaccccatggtgtctttccctgtggccctgtcttgccactgcggcccctgccagatcaagaccaccgactgtggcgtgttcagggaccagcctctggcctgcgctccacaagccagcagcagctctaaggacccccctagccagcccctgaccagcacctctacacctacacctggcgcctccagaagaagcagccaccccctgcccatcaaaacctctgcggcggaggatctgggggaggcggaagcggagggggcggatctaagacccacacctgtcctccatgccctgcccctgaactgctgggcggacctagcgtgttcctgttccccccaaagcccaaggacaccctgatgatcagcaggacccccgaagtgacctgcgtggtggtggatgtgtctcacgaggaccctgaagtgaagttcaattggtacgtggacggcgtggaagtgcacaacgccaagaccaagcccagagaggaacagtacaacagcacctaccgcgtggtgtccgtgctgaccgtgctgcaccaggattggctgaacggcaaagagtacaagtgcaaggtgtccaacaaggccctgcccgctcccatcgaaaagaccatcagcaaggccaagggccagcccagggaaccccaggtgtacacactgccccccagcagggatgagctgaccaagaaccaggtgtccctgacctgtctcgtgaagggcttctacccctccgatatcgccgtggaatgggagagcaacggccagcccgagaacaactacaagaccacaccccccgtgctggacagcgacggctcattcttcctgtacagcaagctgacagtggacaagagcagatggcagcagggcaacgtgttcagctgcagcgtgatgcacgaagccctgcacaaccactacacccagaagtccctgagcctgagccccggcaaataa

SEQ ID NO: 7 - protein, 15 aa, Artificial Sequence
  Synthetic Polypeptide
GGGGSGGGGSGGGGS

(19) United States
(12) Patent Application Publication
(10) Pub. No.: US 2018/0139938 A1
(43) Pub. Date: May 24, 2018

(54) TRANSGENIC PRODUCTION OF FC FUSION PROTEINS

(71) Applicants: Laboratoire Français du Fractionnement et des, Biotechnologies, Les Ulis (FR); BioSourcing SA, Liége (BE)

(72) Inventor: Li-How Chen, Acton, MA (US)

(73) Assignees: Laboratoire Français du Fractionnement et des, Biotechnologies, Les Ulis (FR); GIGA Entreprises, Liége (BE)

(21) Appl. No.: 15/571,604

(22) PCT Filed: May 4, 2016
(86) PCT No.: PCT/IB2016/000683

§ 371 Date: Nov. 3, 2017

Related U.S. Application Data
(60) Provisional application No. 62/156,879, filed on May 4, 2015.

(51) Int. Cl. A01K 67/027; A61K 38/01; C07K 14/59; C12N 9/10

(52) U.S. Cl. CPC A01K 67/0275; A61K 38/018; C07K 14/59; C12N 9/1029; C12N 9/1081; A01K 2217/052; A61K 2039/505; A01K 2217/15; A01K 2227/102; A01K 2227/105; A01K 2227/107; A01K 2267/01; C07K 2319/30; C07K 2319/01

(57) ABSTRACT

In one aspect, the disclosure provides methods, cells and transgenic non-human mammals for the production of fusion proteins comprising one or more polypeptide fused to an Fc domain, as well as the fusion proteins comprising one or more polypeptide fused to an Fc domain obtained from these methods, cells and transgenic non-human mammals.

### RELATED APPLICATIONS

This application claims the benefit under 35 U.S.C. § 119(e) of U.S. provisional application No. 62/156,879, filed May 4, 2015, which is incorporated by reference herein in its entirety.

### FIELD OF INVENTION

The disclosure relates to the field of transgenic production of fusion proteins comprising polypeptides fused to Fc domains.

### BACKGROUND OF INVENTION

Fragment crystallizable (“Fc”) domains correspond to regions of immunoglobulins that bind to cell-surface Fc receptors. Fc domains have been fused to many different proteins. Multiple products comprising Fc-fusion proteins have received FDA approval (e.g., Nulojix (belatacept), Eylea (aflibercept), Arcalyst (rilonacept), NPlate (romiplostim), Orencia (abatacept), Amevive (alefacept), and Enbrel (etanercept); reviewed in Czajkowsky et al. (2012) EMBO Mol. Med. 4:1015-1028.

### SUMMARY OF INVENTION

Described herein are methods for transgenically expressing polypeptides fused to Fc domains, thereby increasing the half-lives of the transgenically produced polypeptides. In one aspect, the disclosure relates to methods of production and use of fusion proteins comprising one or more polypeptide fused to an Fc domain.

Significantly, methods provided herein allow for efficient production of proteins in transgenic animals, including proteins whose expression can be detrimental to animal development. In some aspects, methods described herein allow for improved transgenic expression of multimeric proteins by expressing one or more components of multimeric proteins as fusions with Fc domains. Application of such methods to multimeric proteins can lead to increased half-life and improved protein folding.

Aspects of the invention relate to methods comprising providing a transgenic non-human mammal that has been modified to express a fusion protein comprising one or more polypeptide fused to an Fc domain in the mammary gland, and harvesting the fusion protein from milk produced by the mammary gland of the transgenic non-human mammal. In some embodiments, the Fc domain is a human IgG1 Fc domain. In some embodiments, the sequence of the Fc domain comprises SEQ ID NO:1.

In some embodiments, the fusion protein comprises more than one subunit and the subunits are produced in the same transgenic non-human mammal. In other embodiments, the fusion protein comprises more than one subunit and the subunits are produced in different transgenic non-human mammals. In some embodiments, the subunits are combined after being produced in different transgenic non-human mammals. In some embodiments, the transgenic non-human mammal is bovine, porcine, caprine, ovine or rodent. In some embodiments, the transgenic non-human mammal is a goat. In other embodiments, the transgenic non-human mammal is a rabbit.

In some embodiments, the transgenic non-human mammal has been engineered to recombinantly express a sialyltransferase, such that the fusion protein produced in said mammal has increased sialylation compared to the fusion protein produced in a transgenic non-human mammal that does not express a sialyltransferase. In some embodiments, the fusion protein includes a linker region between the polypeptide and the Fc domain.

Further aspects of the invention provide compositions comprising a fusion protein comprising one or more polypeptide fused to an Fc domain and further comprising milk. In some embodiments, the composition comprises a pharmaceutically acceptable carrier.

Further aspects of the invention provide transgenic non-human mammals that have been modified to express a fusion protein comprising one or more polypeptide fused to an Fc domain. In some embodiments, the transgenic non-human mammal has been modified to express a sialyltransferase. In some embodiments, the transgenic non-human mammal is a bovine, porcine, caprine, ovine or rodent. In some embodiments, the transgenic non-human mammal is a goat. In other embodiments, the transgenic non-human mammal is a rabbit.

Further aspects of the invention relate to methods comprising administering an effective amount of a transgenically produced fusion protein comprising one or more polypeptide fused to an Fc domain to a subject. In some embodiments, the subject is a human or non-human mammal.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF DRAWINGS

The accompanying drawings are not intended to be drawn to scale. The figures are illustrative only and are not required for enablement of the disclosure. For purposes of clarity, not every component may be labeled in every drawing. In the drawings:

FIGS. 1A-1D show schematics of fusion constructs, and nucleic acid and amino acid sequences for eCG-Fc fusion proteins. FIGS. 1A and 1B depict an eCG α subunit-Fc fusion and an eCG β subunit-Fc fusion, respectively. FIGS. 1C and 1D present sequences corresponding to the eCG α subunit-Fc fusion (SEQ ID NO: 3 and 4) and the eCG β subunit-Fc fusion (SEQ ID NO: 5 and 6), respectively.

FIG. 2 depicts a representative Western blot detecting the transient expression of eCG-Fc fusion proteins in 293 cells with an anti-Fc primary antibody.

FIG. 3 depicts a representative Western blot detecting expression of eCG-Fc fusion proteins in the milk of transgenic mice with an anti-Fc primary antibody. The genotype of each mouse is represented by α/-, -/β, or α/β.

FIG. 4 depicts the same representative membrane as FIG. 3 but stained with Ponceau S demonstrating production of the indicated proteins in the milk of transgenic mice. The genotype of each mouse is represented by α/-, -/β, or α/β.

FIGS. 5A and 5B reveal expression of eCG-Fc fusion proteins in the milk of transgenic mice. FIG. 5A presents a representative Ponceau S stained membrane and FIG. 5B presents the same membrane Western blotted using an anti-Fc primary antibody. The genotype of each mouse is represented by α/-, -/β, or α/β, and the generation of the mouse is indicated with F0, F1, or F2.

FIGS. 6A-6C reveal eCG-Fc fusion proteins detected by both an anti-Fc primary antibody and an anti-eCG primary antibody. FIG. 6A presents a representative Western blot using an anti-Fc primary antibody. FIG. 6B presents the same membrane as FIG. 4A but probed with an anti-eCG primary antibody. FIG. 6C presents an overlay of the membrane probed with both an anti-Fc primary antibody and an anti-eCG primary antibody. The genotype of each mouse is represented by α/-, -/β, or α/β.

FIGS. 7A and 7B show relative expression levels of eCG and eCG-fc fusion proteins in the milk of transgenic mice. FIG. 7A presents a representative Western blot using an anti-Fc primary antibody, and FIG. 7B presents a Western blot of the same samples using an anti-eCG primary antibody. Samples were evaluated under non-reducing (NR) and reducing (R) conditions. BME=reducing sample (separate αand β); NR=non reducing sample (not separate α and β).

FIG. 8 presents phenotypes associated with expression of eCG-Fc fusion proteins in the milk of transgenic mice, in a non-limiting embodiment.

### DETAILED DESCRIPTION OF INVENTION

Disclosed herein are methods, cells and transgenic mammals for the production of fusion proteins comprising polypeptides fused to Fc domains. It was surprisingly demonstrated herein that fusing a polypeptide to an Fc domain leads to increased half-life and improved folding of the transgenically produced polypeptide. Methods and compositions associated with the invention allow for increased stability and half-lives of transgenically produced proteins.

Significantly, methods and compositions provided herein address previous challenges in the art related to transgenically expressing in an animal proteins whose expression may be detrimental to development of the animal. In particular, methods described herein address problems associated with transgenically producing proteins in the mammary gland of an animal, when expression of the protein would be expected to have detrimental effects on the development of the animal. In some instances, transgenic expression of a multimeric protein may be detrimental to development of an animal. Using methods described herein, multimeric proteins can be efficiently produced transgenically in the mammary gland of an animal by expressing one or more components of the multimeric protein as fusions with Fc domains. In some embodiments described herein, different components of a multimeric protein are expressed transgenically in different animals.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### Fc Fusions

Aspects of the invention relate to expressing one or more polypeptides fused to a fragment crystallizable (“Fc”) domain. As used herein, an “Fc domain” refers to the portion of an immunoglobulin molecule that interacts with cell surface Fc receptors. An Fc domain can comprise one or more heavy chain constant domains (CH). In some embodiments, the Fc domain comprises two heavy chain constant domains. In some embodiments, the Fc domain comprises heavy chain constant domains CH2 and CH3. Fc domains from immunoglobulins of any isotype (e.g., IgG, IgA, IgM, IgE, IgD) and any subtype (e.g., IgG1, IgG2, IgG3, IgG4) can be compatible with aspects of the invention. In some embodiments, the Fc domain is an IgG1 Fc domain. In some embodiments, the Fc domain is a hybrid Fc domain, as disclosed in and incorporated by reference from U.S. Pat. No. 7,867,491.

Fusion of Fc domains to biologically active proteins is known in the art (see, e.g., U.S. Pat. No. 8,431,132, U.S. Pat. No. 7,867,491, Czajkowsky et al. (2012) EMBO Mol Med 4:1015-1028; Beck et al. (2011) MAbs 3:415-416; Low et al. (2005) Human Reproduction 20(7):1805-1813; Ashkenazi et al. (1993) Int. Rev. Immunol. 10:219-227; Chamow et al. (1996) Trends Biotechnol. 14:52-60; Kim et al. (1994) Eur. J. Immunol. 24:2429-2434. Fc domains can be obtained via routine technology, e.g., PCR amplification from a suitable source. An Fc domain can be naturally occurring or synthetic. In some embodiments, an Fc domain is derived from a human, primate, bovine, porcine, caprine, ovine, rodent or canine mammal. More particularly, an Fc domain is derived from a mammalian source including, without limitation, human or other primate, dog, cat, horse, cow, pig, sheep, goat, rabbit, mouse or rat.

In some embodiments, the Fc domain within an Fc fusion protein that is administered to a human is derived from a human. In other embodiments, the Fc domain within an Fc fusion protein that is administered to a human is derived from a non-human source. In some embodiments, the Fc domain within an Fc fusion protein that is administered to a primate is derived from a primate. In other embodiments, the Fc domain within an Fc fusion protein that is administered to a primate is derived from a non-primate source. In some embodiments, the Fc domain within an Fc fusion protein that is administered to a bovine is derived from a bovine. In other embodiments, the Fc domain within an Fc fusion protein that is administered to a bovine is derived from a non-bovine source. In some embodiments, the Fc domain within an Fc fusion protein that is administered to a porcine is derived from a porcine. In other embodiments, the Fc domain within an Fc fusion protein that is administered to a porcine is derived from a non-porcine source. In some embodiments, the Fc domain within an Fc fusion protein that is administered to a caprine is derived from a caprine. In other embodiments, the Fc domain within an Fc fusion protein that is administered to a caprine is derived from a non-caprine source. In some embodiments, the Fc domain within an Fc fusion protein that is administered to a ovine is derived from a ovine. In other embodiments, the Fc domain within an Fc fusion protein that is administered to a ovine is derived from a non-ovine source. In some embodiments, the Fc domain within an Fc fusion protein that is administered to a rodent is derived from a rodent. In other embodiments, the Fc domain within an Fc fusion protein that is administered to a rodent is derived from a non-rodent source. In some embodiments, the Fc domain within an Fc fusion protein that is administered to a dog is derived from a dog. In other embodiments, the Fc domain within an Fc fusion protein that is administered to a dog is derived from a non-dog source. In some embodiments, the Fc domain within an Fc fusion protein that is administered to a cat is derived from a cat. In other embodiments, the Fc domain within an Fc fusion protein that is administered to a cat is derived from a non-cat source. In some embodiments, the Fc domain within an Fc fusion protein that is administered to a horse is derived from a horse. In other embodiments, the Fc domain within an Fc fusion protein that is administered to a horse is derived from a non-horse source. In some embodiments, the Fc domain within an Fc fusion protein that is administered to a cow is derived from a cow. In other embodiments, the Fc domain within an Fc fusion protein that is administered to a cow is derived from a non-cow source. In some embodiments, the Fc domain within an Fc fusion protein that is administered to a pig is derived from a pig. In other embodiments, the Fc domain within an Fc fusion protein that is administered to a pig is derived from a non-pig source. In some embodiments, the Fc domain within an Fc fusion protein that is administered to a sheep is derived from a sheep. In other embodiments, the Fc domain within an Fc fusion protein that is administered to a sheep is derived from a non-sheep source. In some embodiments, the Fc domain within an Fc fusion protein that is administered to a goat is derived from a goat. In other embodiments, the Fc domain within an Fc fusion protein that is administered to a goat is derived from a non-goat source. In some embodiments, the Fc domain within an Fc fusion protein that is administered to a rabbit is derived from a rabbit. In other embodiments, the Fc domain within an Fc fusion protein that is administered to a rabbit is derived from a non-rabbit source. In some embodiments, the Fc domain within an Fc fusion protein that is administered to a mouse is derived from a mouse. In other embodiments, the Fc domain within an Fc fusion protein that is administered to a mouse is derived from a non-mouse source. In some embodiments, the Fc domain within an Fc fusion protein that is administered to a rat is derived from a rat. In other embodiments, the Fc domain within an Fc fusion protein that is administered to a rat is derived from a non-rat source.

In some embodiments, the Fc domain comprises the sequence of SEQ ID NO:1. In certain embodiments, the Fc domain consists of the sequence of SEQ ID NO:1. In some embodiments, the Fc domain is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO:1.

The amino acid sequence of a non-limiting example of an Fc domain is provided in SEQ ID NO: 1:

KTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRW
QQGNVFSCSVMHEALHNHYTQKSLSLSPGK

The nucleic acid sequence of a non-limiting example of an Fc domain is provided in SEQ ID NO: 2:

AAGACCCACACCTGTCCTCCCTGTCCCGCCCCTGAACTGCTGGGAGGCC
CTAGCGTGTTCCTGTTCCCCCCAAAGCCCAAGGACACCCTGATGATCAG
CAGGACCCCCGAAGTGACCTGCGTGGTGGTGGATGTGTCTCACGAGGAC
CCTGAAGTGAAGTTCAATTGGTACGTGGACGGCGTGGAAGTGCACAACG
CCAAGACCAAGCCCAGAGAGGAACAGTACAACAGCACCTACAGGGTGGT
GTCCGTGCTGACCGTGCTGCACCAGGATTGGCTGAACGGCAAAGAGTAC
AAGTGCAAGGTGTCCAACAAGGCCCTGCCTGCCCCCATCGAAAAGACCA
TCAGCAAGGCCAAGGGCCAGCCCAGGGAACCCCAGGTGTACACACTGCC
CCCCAGCAGGGATGAGCTGACCAAGAACCAGGTGTCCCTGACCTGTCTC
GTGAAGGGCTTCTACCCCTCCGATATCGCCGTGGAATGGGAGAGCAACG
GCCAGCCCGAGAACAACTACAAGACCACCCCCCCTGTGCTGGACAGCGA
CGGCTCATTCTTCCTGTACAGCAAGCTGACAGTGGACAAGAGCAGATGG
CAGCAGGGCAACGTGTTCAGCTGCAGCGTGATGCACGAGGCCCTGCACA
ACCACTACACCCAGAAGTCCCTGAGCCTGAGCCCCGGCAAATAATGA

In some embodiments, the nucleic acid encoding the Fc domain comprises SEQ ID NO:2. In certain embodiments, the nucleic acid encoding the Fc domain consists of SEQ ID NO:2. In some embodiments, the nucleic acid sequence encoding the Fc domain is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO:2.

The Fc domain can be covalently linked to a polypeptide. In some embodiments, the polypeptide is attached directly to the Fc domain. For example, a polypeptide can be attached to the flexible hinge region of the Fc domain. A linker region can also be included connecting the polypeptide to the Fc domain, as would be understood by one of ordinary skill in the art. An example of a linker sequence is provided by SEQ ID NO: 7:

GGGGSGGGGSGGGGS

In some aspects, methods provided herein may be advantageous for production of multimeric proteins in a transgenic non-human mammal. As used herein, a “multimeric protein” refers to a protein that is comprised of more than one independent, non-covalently linked, subunit or polypeptide that can combine to form a single protein. In some embodiments, each subunit of a multimeric protein is fused to an Fc domain. In some embodiments, at least one subunit of a multimeric protein is fused to an Fc domain. In some embodiments, each subunit of a multimeric protein is produced in a transgenic non-human mammal and combined following the harvesting of each subunit from the respective transgenic mammal. One or more subunits of a multimeric protein can be produced in the same transgenic animal or in different transgenic animals.

An Fc domain associated with the invention may comprise one or more N-glycans at the Fc-gamma glycosylation site in the heavy chain (Asn297) of the Fc fragment. A variety of glycosylation patterns can occur at the Fc gamma glycosylation site. Oligosaccharides found at this site include galactose, N-acetylglucosamine (GlcNac), mannose, sialic acid, N-acetylneuraminic acid (NeuAc or NANA), N-glycolylneuraminic (NGNA) and fucose. N-glycans found at the Fc gamma glycosylation site generally have a common core structure consisting of an unbranched chain of a first N-acetylglucosamine (GlcNAc), which is attached to the asparagine of the antibody, a second GlcNAc that is attached to the first GlcNac and a first mannose that is attached to the second GlcNac. Two additional mannoses are attached to the first mannose of the GlcNAc-GlcNAc-mannose chain to complete the core structure and providing two “arms” for additional glycosylation. In addition, fucose residues can be attached to the N-linked first GlcNAc.

Aspects of the invention relate to fusion of one or more polypeptides to an Fc domain. The Fc domain can be fused at either the N or C terminus of the polypeptide. In some embodiments, the Fc domain is fused to the C terminus of the polypeptide. In some embodiments, two or more subunits of a multimeric protein are each fused to an Fc domain. The polypeptide fused to an Fc domain can be produced in the mammary gland of a transgenic mammal. Multiple polypeptides, including subunits of polypeptides or multimeric proteins, fused to Fc domains can be produced in the mammary gland of the same transgenic mammal or in mammary glands of different transgenic mammals and combined following harvest of each subunit from the respective transgenic mammal.

It should be appreciated that methods, cells and compositions described herein can be compatible with any polypeptide that is fused to an Fc domain.

Purification from Transgenic Animals

In one aspect, a fusion protein comprising a polypeptide fused to an Fc domain is purified from transgenic non-human mammals. In some embodiments, the fusion protein comprising a polypeptide fused to an Fc domain is secreted into the milk of the transgenic non-human mammals. In some embodiments, two or more subunits of a multimeric protein are each fused to an Fc domain. In some embodiments, the subunits fused to an Fc domain are secreted into the milk of the same or different transgenic non-human mammals. The fusion protein comprising a polypeptide fused to an Fc domain can be purified from the milk of transgenic non-human mammals such that the fusion protein comprising a polypeptide fused to an Fc domain is substantially pure. In some embodiments, each subunit of a multimeric protein fused to an Fc domain can be purified from the milk of the same or different transgenic non-human mammals such that each subunit fused to an Fc domain is substantially pure. In such embodiments, the subunits fused to an Fc domain can be subsequently combined. In some embodiments, substantially pure includes substantially free of contaminants.

In one aspect, the fusion protein comprising one or more polypeptide fused to an Fc domain is purified from a mammary epithelial cell that has been modified to express the fusion protein comprising one or more polypeptide fused to an Fc domain. The fusion protein comprising a polypeptide fused to an Fc domain can be purified from a mammary epithelial cell such that the fusion protein comprising a polypeptide fused to an Fc domain is substantially pure. In some embodiments, each subunit of a multimeric protein fused to an Fc domain can be purified from the same or different mammary epithelial cells such that each subunit fused to an Fc domain is substantially pure. In some embodiments, substantially pure includes substantially free of contaminants.

A fusion protein comprising a polypeptide fused to an Fc domain that is harvested from the milk of a transgenic non-human mammal or from a mammary epithelial cell can be purified using any suitable means known in the art. In some embodiments, the fusion protein comprising a polypeptide fused to an Fc domain is purified using column chromatography. Column chromatography is well known in the art (see, e.g., Current Protocols in Essential Laboratory Techniques Unit 6.2 (2008) for general chromatography methods). In some embodiments, the fusion protein comprising a polypeptide fused to an Fc domain is purified using protein-G/A affinity chromatography (see, e.g., Carter (2011) Exp Cell Res 317:1261-1269). In some embodiments, the fusion protein comprising a polypeptide fused to an Fc domain is purified by immunoprecipitation (see, e.g., Current Protocols in Cell Biology Unit 7.2 (2001)). In some embodiments, the fusion protein comprising a polypeptide fused to an Fc domain is purified with an antibody or fragment thereof that specifically recognizes the polypeptide or with an antibody or fragment thereof that specifically recognizes the Fc domain.

### Constructs for the Generation of Transgenic Animals Expressing Fusion Proteins

In some embodiments, to produce primary cell lines containing a construct (e.g., encoding a fusion protein comprising one or more polypeptide fused to an Fc domain) for use in producing transgenic goats by nuclear transfer, the constructs can be transfected into primary goat skin epithelial cells, which are clonally expanded and fully characterized to assess transgene copy number, transgene structural integrity and chromosomal integration site. As used herein, “nuclear transfer” refers to a method of cloning wherein the nucleus from a donor cell is transplanted into an enucleated oocyte.

Coding sequences for proteins of interest (e.g., a fusion protein comprising a polypeptide fused to an Fc domain) can be obtained from any suitable source including by screening libraries of genomic material or reverse-translated messenger RNA derived from the animal of choice, obtained from sequence databases such as NCBI, Genbank, or by obtaining the sequences of the polypeptide. The sequences can be cloned into an appropriate plasmid vector and amplified in a suitable host organism, like E. coli. After amplification of the vector, the DNA construct can be excised, purified from the remains of the vector and introduced into expression vectors that can be used to produce transgenic animals. The transgenic animals will have the desired transgenic protein integrated into their genome.

After amplification of the vector, the DNA construct can also be excised with the appropriate 5′ and 3′ control sequences, purified away from the remains of the vector and used to produce transgenic animals that have integrated into their genome the desired expression constructs. Conversely, with some vectors, such as yeast artificial chromosomes (YACs), it is not necessary to remove the assembled construct from the vector; in such cases the amplified vector may be used directly to make transgenic animals. The coding sequence can be operatively linked to a control sequence, which enables the coding sequence to be expressed in the milk of a transgenic non-human mammal.

A DNA sequence which is suitable for directing production of a fusion protein comprising a polypeptide fused to an Fc domain, to the milk of transgenic animals can carry a 5′-promoter region derived from a naturally-derived milk protein. This promoter is consequently under the control of hormonal and tissue-specific factors and is most active in lactating mammary tissue. In some embodiments, the promoter is a caprine beta casein promoter. The promoter can be operably linked to a DNA sequence directing the production of a protein leader sequence, which directs the secretion of the transgenic protein across the mammary epithelium into the milk. In some embodiments, a 3′-sequence, which can be derived from a naturally secreted milk protein, can be added to improve stability of mRNA.

As used herein, a “leader sequence” or “signal sequence” is a nucleic acid sequence that encodes a protein secretory signal, and, when operably linked to a downstream nucleic acid molecule encoding a transgenic protein directs secretion. The leader sequence may be the native human leader sequence, an artificially-derived leader, or may obtained from the same gene as the promoter used to direct transcription of the transgene coding sequence, or from another protein that is normally secreted from a cell, such as a mammalian mammary epithelial cell.

In some embodiments, the promoters are milk-specific promoters. As used herein, a “milk-specific promoter” is a promoter that naturally directs expression of a gene in a cell that secretes a protein into milk (e.g., a mammary epithelial cell) and includes, for example, the casein promoters, e.g., α-casein promoter (e.g., alpha S-1 casein promoter and alpha S2-casein promoter), β-casein promoter (e.g., the goat beta casein gene promoter (DiTullio, BIOTECHNOLOGY 10:74-77, 1992), γ-casein promoter, κ-casein promoter, whey acidic protein (WAP) promoter (Gordon et al., BIOTECHNOLOGY 5: 1183-1187, 1987), β-lactoglobulin promoter (Clark et al., BIOTECHNOLOGY 7: 487-492, 1989) and α-lactalbumin promoter (Soulier et al., FEBS LETTS. 297:13, 1992). Also included in this definition are promoters that are specifically activated in mammary tissue, such as, for example, the long terminal repeat (LTR) promoter of the mouse mammary tumor virus (MMTV).

As used herein, a coding sequence and regulatory sequences are said to be “operably joined” when they are covalently linked in such a way as to place the expression or transcription of the coding sequence under the influence or control of the regulatory sequences. In order for the coding sequences to be translated into a functional protein the coding sequences are operably joined to regulatory sequences. Two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region is operably joined to a coding sequence if the promoter region were capable of effecting transcription of that DNA sequence such that the resulting transcript might be translated into the desired protein or polypeptide.

As used herein, a “vector” may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to, plasmids and phagemids. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium, or just a single time per host as the host reproduces by mitosis. In the case of phage, replication may occur actively during a lytic phase or passively during a lysogenic phase. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells, which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase or alkaline phosphatase), and genes which visibly affect the phenotype of transformed or transfected cells, hosts, colonies or plaques. Preferred vectors are those capable of autonomous replication and expression of the structural gene products present in the DNA segments to which they are operably joined.

### Mammary Epithelial Cells and Transgenic Animals for Production of Fusion Proteins

In one aspect, the disclosure provides mammary gland epithelial cells that express fusion proteins comprising one or more polypeptide fused to an Fc domain. In some embodiments, the disclosure provides a transgenic non-human mammal comprising mammary gland epithelial cells that express fusion proteins comprising one or more polypeptide fused to an Fc domain

In one aspect, the disclosure provides a method for the production of fusion proteins comprising one or more polypeptide fused to an Fc domain, comprising (a) transfecting non-human mammalian cells with a transgene DNA construct encoding a fusion protein comprising one or more polypeptide fused to an Fc domain; (b) selecting cells in which said transgene DNA construct has been inserted into the genome of the cells; and (c) performing a first nuclear transfer procedure to generate a non-human transgenic mammal heterozygous for the fusion protein comprising one or more polypeptide fused to an Fc domain, and that can express the fusion protein comprising one or more polypeptide fused to an Fc domain in its milk.

In one aspect, the disclosure provides a method of (a) providing a non-human transgenic mammal engineered to express a fusion protein comprising one or more polypeptide fused to an Fc domain, (b) expressing the fusion protein comprising one or more polypeptide fused to an Fc domain, in the milk of the non-human transgenic mammal; and (c) isolating the fusion protein comprising one or more polypeptide fused to an Fc domain, produced in the milk.

Transgenic animals can also be generated according to methods known in the art (See e.g., U.S. Pat. No. 5,945,577). Animals suitable for transgenic expression, include, but are not limited to goat, sheep, bison, camel, cow, rabbit, pig, horse, rat or llama. Suitable animals also include bovine, caprine, porcine, rodent and ovine, which relate to various species of cows, goats, pig, rat and sheep, respectively. Suitable animals also include ungulates. As used herein, “ungulate” is of or relating to a hoofed typically herbivorous quadruped mammal, including, without limitation, sheep, goats, cattle and horses. In one embodiment, the animals are generated by co-transfecting primary cells with separate constructs. These cells are then used for nuclear transfer. Alternatively, if micro-injection is used to generate the transgenic animals, the constructs may be injected.

Cloning will result in a multiplicity of transgenic animals—each capable of producing a fusion protein comprising one or more polypeptide fused to an Fc domain or other gene construct of interest. The production methods include the use of the cloned animals and the offspring of those animals. In some embodiments, the cloned animals are caprines, bovines. Cloning also encompasses the nuclear transfer of fetuses, nuclear transfer, tissue and organ transplantation and the creation of chimeric offspring.

One step of the cloning process comprises transferring the genome of a cell that contains the transgene encoding the fusion protein comprising one or more polypeptide fused to an Fc domain into an enucleated oocyte. As used herein, “transgene” refers to any piece of a nucleic acid molecule that is inserted by artifice into a cell, or an ancestor thereof, and becomes part of the genome of an animal which develops from that cell. Such a transgene may include a gene which is partly or entirely exogenous (i.e., foreign) to the transgenic animal, or may represent a gene having identity to an endogenous gene of the animal.

Suitable mammalian sources for oocytes include goats, sheep, cows, rabbits, guinea pigs, hamsters, rats, non-human primates, etc. Preferably, oocytes are obtained from ungulates, and most preferably goats or cattle. Methods for isolation of oocytes are well known in the art. Essentially, the process comprises isolating oocytes from the ovaries or reproductive tract of a mammal, e.g., a goat. A readily available source of ungulate oocytes is from hormonally-induced female animals. For the successful use of techniques such as genetic engineering, nuclear transfer and cloning, oocytes may preferably be matured in vivo before these cells may be used as recipient cells for nuclear transfer, and before they were fertilized by the sperm cell to develop into an embryo. Metaphase II stage oocytes, which have been matured in vivo, have been successfully used in nuclear transfer techniques. Essentially, mature metaphase II oocytes are collected surgically from either non-super ovulated or super ovulated animals several hours past the onset of estrus or past the injection of human chorionic gonadotropin (hCG) or similar hormone.

One of the tools used to predict the quantity and quality of the recombinant protein expressed in the mammary gland is through the induction of lactation (Ebert K M, 1994). Induced lactation allows for the expression and analysis of protein from the early stage of transgenic production rather than from the first natural lactation resulting from pregnancy, which is at least a year later. Induction of lactation can be done either hormonally or manually.

In some embodiments, the compositions of fusion proteins produced according to the methods provided herein further comprise milk. In some embodiments, the methods provides herein includes a step of isolating the fusion proteins from the milk of a transgenic animal (See e.g., Pollock et al., Journal of Immunological Methods, Volume 231, Issues 1-2, 10 Dec. 1999, Pages 147-157).

Thus, in one aspect the disclosure provides mammary gland epithelial cells and transgenic non-human mammals that produce a fusion protein comprising one or more polypeptide fused to an Fc domain. Mammary gland epithelial cells and transgenic non-human mammals according to aspects of the invention express nucleic acid sequences encoding a fusion protein comprising one or more polypeptide fused to an Fc domain.

### Production of Fusion Proteins

In one aspect, a fusion protein comprising one or more polypeptide fused to an Fc domain produced as described herein in transgenic non-human mammals or in mammary epithelial cells has altered characteristics compared to the same fusion protein produced by other methods. For example, fusion proteins produced as described herein can exhibit increased half-lives and/or stability compared to fusion proteins produced by other methods. Fusion proteins produced as described herein can also exhibit decreased immunogenicity compared to fusion proteins produced by other methods.

In one aspect, the disclosure provides recombinant or transgenically produced fusion proteins comprising one or more polypeptide fused to an Fc domain and compositions comprising such proteins wherein the fusion proteins exhibit glycosylation and/or sialylation. For example, the fusion proteins produced using methods described herein may exhibit comparable or higher levels of glycosylation and/or sialylation than the same fusion proteins produced by other methods, including other recombinant methods.

For example, in some embodiments, a fusion protein comprising one or more polypeptide fused to an Fc domain produced in mammary epithelial cells of a non-human mammal may have increased levels of glycosylation and/or sialylation when compared to the same fusion protein not produced in mammary gland epithelial cells. In some embodiments, the fusion protein not produced in mammary gland epithelial cells is produced in cell culture. As used herein, “produced in cell culture” when compared to fusion proteins produced in mammary epithelial cells, refers to fusion proteins produced in standard production cell lines (e.g., CHO cells or baculovirus-Sf9 cells) but excluding mammary epithelial cells.

In some embodiments the methods above further comprise steps for inducing lactation. In still other embodiments the methods further comprise additional isolation and/or purification steps. In yet other embodiments the methods further comprise steps for comparing the glycosylation pattern of the fusion protein produced in cell culture, e.g. non-mammary cell culture. In further embodiments, the methods further comprise steps for comparing the glycosylation pattern of the fusion protein obtained to fusion proteins produced by non-mammary epithelial cells. Such cells can be cells of a cell culture. Experimental techniques for assessing the glycosylation pattern of fusion proteins are known to those of ordinary skill in the art. Such methods include, e.g., liquid chromatography mass spectrometry, tandem mass spectrometry, and Western blot analysis.

In one aspect, the fusion protein comprising one or more polypeptide fused to an Fc domain disclosed herein is generated by producing the fusion protein comprising one or more polypeptide fused to an Fc domain in a transgenic non-human mammal or in mammary epithelial cells. In some embodiments, it may be advantageous to increase the sialylation level of the fusion protein comprising one or more polypeptide fused to an Fc domain. The sialylation levels of the fusion protein comprising one or more polypeptide fused to an Fc domain can be increased for instance by subjecting the fusion protein to sialyltransferases. The fusion protein comprising one or more polypeptide fused to an Fc domain can be subjected to sialyltransferases in vitro or in vivo. The fusion protein comprising one or more polypeptide fused to an Fc domain can be sialylated in vitro by subjecting the fusion protein to a sialyltransferase and the appropriate saccharide based substrate. The fusion protein comprising one or more polypeptide fused to an Fc domain can be sialylated in vivo by producing a sialyltransferase in the mammary gland or mammary epithelial cells.

In one aspect, the disclosure provides methods for the production in the mammary gland of transgenic animals and mammary epithelial cells of fusion proteins comprising one or more polypeptide fused to an Fc domain with increased levels of alpha-2,6-sialylation. In some embodiments, fusion proteins that exhibits increased sialylation may exhibit increased anti-inflammatory properties.

In one aspect, the disclosure provides transgenic animals (and mammary epithelial cells) that are transgenic for the production in the mammary gland of a fusion protein comprising one or more polypeptide fused to an Fc domain and that are transgenic for the production of sialyltransferase. The fusion proteins comprising one or more polypeptide fused to an Fc domain produced by such animals and cells are expected to have increased levels of terminal alpha-2,6-sialic acid linkages. In some embodiments, the transgenic animals (and mammary epithelial cells) are transgenic for the production in the mammary gland of a fusion protein comprising one or more polypeptide fused to an Fc domain and are transgenic for the production of sialyltransferase.

In one aspect, the disclosure provides methods of treating a subject comprising administering to a subject the fusion protein comprising one or more polypeptide fused to an Fc domain that has increased levels of terminal alpha-2,6-sialic acid linkages.

The fusion protein comprising one or more polypeptide fused to an Fc domain can be obtained, in some embodiments, by harvesting the fusion protein comprising one or more polypeptide fused to an Fc domain from the milk of a transgenic animal produced as provided herein or from an offspring of said transgenic animal. In some embodiments the fusion protein comprising one or more polypeptide fused to an Fc domain produced by the transgenic mammal is produced at a level of at least 1 gram per liter of milk produced, preferably at least 2, 3, 4 grams per liter of milk produced and preferably at least 5 grams per liter of milk produced.

For example, in some embodiments, methods described herein allow for production of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 grams per liter.

### Compositions

In some aspect, the disclosure provides compositions, including pharmaceutical compositions, which comprise fusion proteins comprising one or more polypeptide fused to an Fc domain and a pharmaceutically acceptable vehicle, diluent or carrier. In some embodiments, the compositions comprise milk.

In some embodiments, the compositions provided are employed for in vivo applications. Depending on the intended mode of administration in vivo the compositions used may be in the dosage form of solid, semi-solid or liquid such as, e.g., tablets, pills, powders, capsules, gels, ointments, liquids, suspensions, or the like. Preferably, the compositions are administered in unit dosage forms suitable for single administration of precise dosage amounts. The compositions may also include, depending on the formulation desired, pharmaceutically acceptable carriers or diluents, which are defined as aqueous-based vehicles commonly used to formulate pharmaceutical compositions for animal or human administration. The diluent is selected so as not to affect the biological activity of the human recombinant protein of interest. Examples of such diluents are distilled water, physiological saline, Ringer's solution, dextrose solution, and Hank's solution. The same diluents may be used to reconstitute a lyophilized recombinant protein of interest. In addition, the pharmaceutical composition may also include other medicinal agents, pharmaceutical agents, carriers, adjuvants, nontoxic, non-therapeutic, non-immunogenic stabilizers, etc. Effective amounts of such diluent or carrier are amounts which are effective to obtain a pharmaceutically acceptable formulation in terms of solubility of components, biological activity, etc. In some embodiments the compositions provided herein are sterile.

Administration during in vivo treatment may be by any number of routes, including oral, parenteral, intramuscular, intranasal, sublingual, intratracheal, inhalation, ocular, vaginal, and rectal. Intracapsular, intravenous, and intraperitoneal routes of administration may also be employed. The skilled artisan recognizes that the route of administration varies depending on the response desired. For example, the compositions herein may be administered to a subject via oral, parenteral or topical administration. In one embodiment, the compositions herein are administered by intravenous infusion.

The compositions, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compositions in water soluble form. Additionally, suspensions of the active compositions may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compositions to allow for the preparation of highly concentrated solutions. Alternatively, the active compositions may be in powder form for constitution with a suitable vehicle, e.g., sterile pyrogen-free water, before use.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

The component or components may be chemically modified so that oral delivery is efficacious. Generally, the chemical modification contemplated is the attachment of at least one molecule, where said molecule permits (a) inhibition of proteolysis; and (b) uptake into the blood stream from the stomach or intestine. Also desired is the increase in overall stability and increase in circulation time in the body. Examples of such molecules include: polyethylene glycol, copolymers of ethylene glycol and propylene glycol, carboxymethyl cellulose, dextran, polyvinyl alcohol, polyvinyl pyrrolidone and polyproline. Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189. Other polymers that could be used are poly-1,3-dioxolane and poly-1,3,6-tioxocane. Preferred for pharmaceutical usage, as indicated above, are polyethylene glycol molecules. For oral compositions, the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the biologically active material or by release of the biologically active material beyond the stomach environment, such as in the intestine. For buccal administration, the compositions may take the form of tablets or lozenges formulated in conventional manner.

The compositions may also be formulated in rectal or vaginal compositions such as suppositories or retention enemas, e.g., containing conventional suppository bases such as cocoa butter or other glycerides.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compositions, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

Therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the disclosure contain an effective amount of a fusion protein comprising one or more polypeptide fused to an Fc domain and optionally therapeutic agents included in a pharmaceutically-acceptable carrier. The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compositions of the present disclosure, and with each other, in a manner such that there is no interaction which would substantially impair the desired pharmaceutical efficiency.

The therapeutic agent(s), including fusion proteins, may in some embodiments be provided in particles. Particles as used herein means nano or microparticles (or in some instances larger) which can consist in whole or in part of the therapeutic agent or can include other additional therapeutic agents. The particle may include, in addition to the therapeutic agent(s), any of those materials routinely used in the art of pharmacy and medicine, including, but not limited to, erodible, non erodible, biodegradable, or non biodegradable material or combinations thereof. The particles may be microcapsules which contain the therapeutic agent in a solution or in a semi-solid state. The particles may be of virtually any shape.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The methods and techniques of the present disclosure are generally performed according to conventional methods well-known in the art. Generally, nomenclatures used in connection with, and techniques of biochemistry, enzymology, molecular and cellular biology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teaching that is referenced hereinabove. However, the citation of any reference is not intended to be an admission that the reference is prior art.

### EXAMPLES

### Example 1

### Generation of Transgenic Mice that Produce eCG

Transgenic mice were generated that include nucleic acid sequences encoding the α and β subunits of eCG in their genome. The mice producing eCG were generated using traditional microinjection techniques. The cDNA encoding the α and β subunits of eCG was synthesized based on the published amino acid sequences. These DNA sequences were ligated into an expression vector. In these plasmids, the nucleic acid sequence encoding eCG is under control of a promoter facilitating the expression of eCG in the mammary glands of the mice. The prokaryotic sequences were removed and the DNA microinjected into pre-implantation embryos of the mice. These embryos were then transferred to pseudo pregnant females. The progeny that resulted were screened for the presence of the transgenes. Those that carried the transgenes of both the α and β subunits of eCG were identified as transgenic founders.

When age appropriate, the founder animals were bred to produce F1 progeny. Following pregnancy and parturition, the mice were milked. Production of eCG in the milk of the transgenic mice was quantified using a PMSG (eCG) ELISA kit (DRG International), as presented in Table 1. High eCG-expressing F1 mice were bred when age appropriate to produce F2 mice.

TABLE 1
Transgenic expression of eCG in mice

| Founder | F1 | F2 | IU/mL by ELISA |
|---|---|---|---|
| 93 | | | 1152 |
| | 239 | | 632 |
| | 244 | | 222 |
| 129 | | | 1758 |
| | 284 | | 727 |
| 153 | | | 1662 |
| | 251 | | 14250 |
| | | 429 | 3203 |
| 13 | | | |
| | 327 | | 15261 |
| 78 | | | |
| | 235 | | 19350 |
| 150 | | | |
| | 312 | | 12491 |

### Example 2

### Generation of Transgenic ST6 Mice that Produce eCG

In order to increase the sialylation levels of the α and β subunits of eCG produced in transgenic mice, the eCG-expressing mice described in Example 1 were crossed with mice that were transgenic for the production of a sialyltransferase (ST). As shown in Table 2, an initial cross between transgenic eCG-producing mice and transgenic ST3Gal6-producing mice resulted in 24 progeny mice that had both eCG and ST3Gal6 transgenes. When age appropriate, these mice were bred. Following pregnancy and parturition, they were milked and the eCG produced in milk of the animals was characterized.

TABLE 2
Generation of crossing eCG- and ST-producing mice

| | eCG | — |
|---|---|---|
| ST | 24 (7 males, 17 females) | 22 (9 males, 13 females) |
| — | 22 (9 males, 13 females) | 21 |

### Example 3

### Generation and Expression of eCG-Fc Fusions

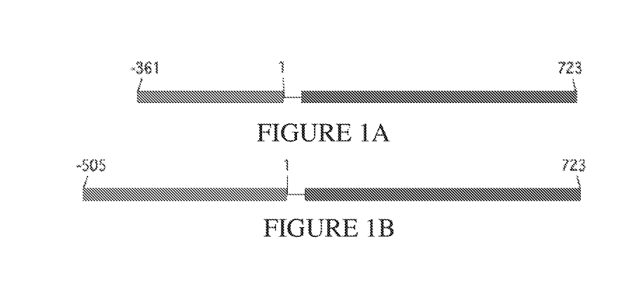

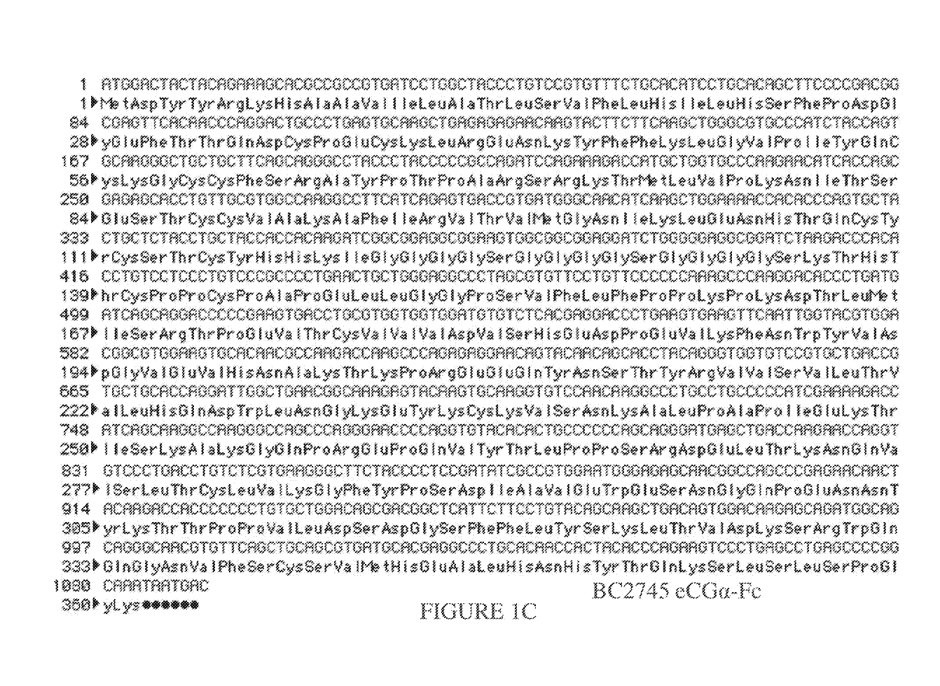

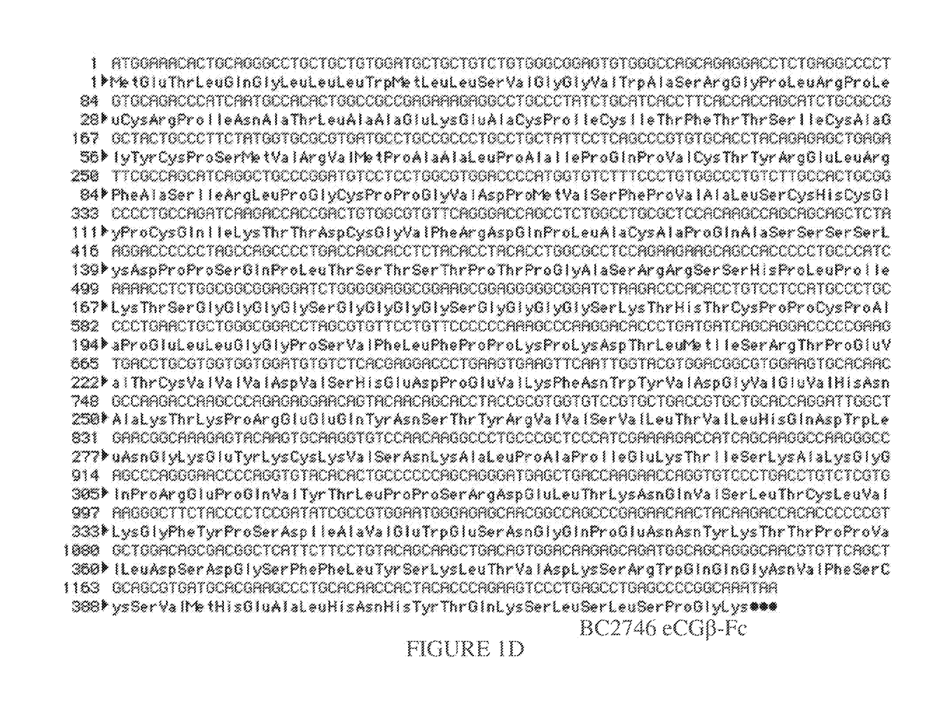

To increase the half-life of the transgenically produced eCG, constructs were generated in which both of the eCG subunits were C-terminally fused to the human IgG1 Fc sequence. (FIG. 1A-1D). The constructs were transfected into 293 tissue culture cells either independently or together. Supernatant from the transfected cells was collected and analyzed for eCG production by ELISA, as presented in Table 3.

TABLE 3
Transient expression of eCG-Fc fusion
in the supernatant of 293 cells

| Supernatant # | Transfected with | IU/mL by ELISA | ELISA sample # |
|---|---|---|---|
| 1 | α-Fc/β-Fc | 3.3 | 18 |
| 2 | α-Fc/β | 1.3 | 19 |
| 3 | α/β-Fc | 4.8 | 20 |
| 4 | α/β | 16.9 | 21 |
| 0 | Mock | 1.5 | 22 |

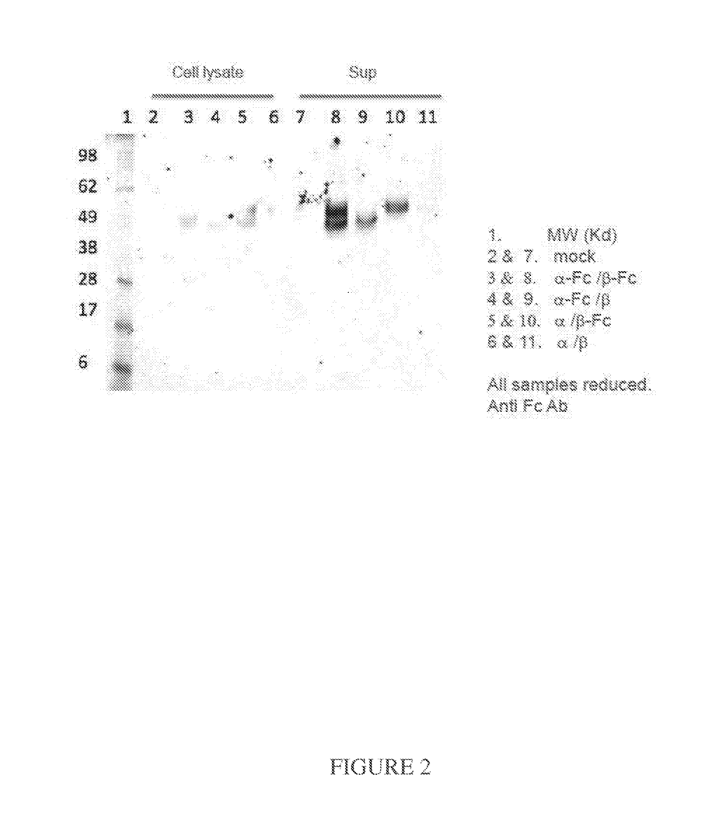

Supernatants and cell lysates were also collected from the transfected cells and analyzed by Western blot, as shown in FIG. 2. The samples were probed with an anti-Fc primary antibody. eCG subunits were detected by Western blot in the supernatant of cells expressing either subunit or both subunits fused to the Fc region (lanes 8, 9, and 10), but not in the supernatant of cells that expressed the eCG subunits without the Fc fusion (lane 11).

The Sequences depicted in FIG. 1C correspond to SEQ ID NOs: 3 and 4, which correspond to the following sequences:

SEQ ID NO: 3:
MDYYRKHAAVILATLSVFLHILHSFPDGEFTTQDCPECKLRENKYFFKLG
VPIYQCKGCCFSRAYPTPARSRKTMLVPKNITSESTCCVAKAFIRVTVMG
NIKLENHTQCYCSTCYHHKIGGGGSGGGGSGGGGSKTHTCPPCPAPELLG
GPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKII
SKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHY
TQKSLSLSPGK
SEQ ID NO: 4:
ATGGACTACTACAGAAAGCACGCCGCCGTGATCCTGGCTACCCTGTCCGT
GTTTCTGCACATCCTGCACAGCTTCCCCGACGGCGAGTTCACAACCCAGG
ACTGCCCTGAGTGCAAGCTGAGAGAGAACAAGTACTTCTTCAAGCTGGGC
GTGCCCATCTACCAGTGCAAGGGCTGCTGCTTCAGCAGGGCCTACCCTAC
CCCCGCCAGATCCAGAAAGACCATGCTGGTGCCCAAGAACATCACCAGCG
AGAGCACCTGTTGCGTGGCCAAGGCCTTCATCAGATGGACCGTGATGGGC
AACATCAAGCTGGAAAACCACACCCAGTGCTACTGCTCTACCTGCTACCA
CCACAAGATCGGCGGAGGCGGAAGTGGCGGCGGAGGATCTGGGGGAGGCG
GATCTAAGACCCACACCTGTCCTCCCTGTCCCGCCCCTGAACTGCTGGGA
GGCCCTAGCGTGTTCCTGTTCCCCCCAAAGCCCAAGGACACCCTGATGAT
CAGCAGGACCCCCGAAGTGACCTGCGTGGTGGTGGATGTGTCTCACGAGG
ACCCTGAAGTGAAGTTCAATTGGTACGTGGACGGCGTGGAAGTGCACAAC
GCCAAGACCAAGCCCAGAGAGGAACAGTACAACAGCACCTACAGGGTGGT
GTCCGTGCTGACCGTGCTGCACCAGGATTGGCTGAACGGCAAAGAGTACA
AGTGCAAGGTGTCCAACAAGGCCCTGCCTGCCCCCATCGAAAAGACCATC
AGCAAGGCCAAGGGCCAGCCCAGGGAACCCCAGGTGTACACACTGCCCCC
CAGCAGGGATGAGCTGACCAAGAACCAGGTGTCCCTGACCTGTCTCGTGA
AGGGCTTCTACCCCTCCGATATCGCCGTGGAATGGGAGAGCAACGGCCAG
CCCGAGAACAACTACAAGACCACCCCCCCTGTGCTGGACAGCGACGGCTC
ATTCTTCCTGTACAGCAAGCTGACAGTGGACAAGAGCAGATGGCAGCAGG
GCAACGTGTTCAGCTGCAGCGTGATGCACGAGGCCCTGCACAACCACTAC
ACCCAGAAGTCCCTGAGCCTGAGCCCCGGCAAATAATGA

The Sequences depicted in FIG. 1D correspond to SEQ ID NOs: 5 and 6, which correspond to the following sequences:

SEQ ID NO: 5:
METLQGLLLWMLLSVGGVWASRGPLRPLCRPINATLAAEKEACPICITFT
TSICAGYCPSMVRVMPAALPAIPQPVCTYRELRFASIRLPGCPPGVDPMV
SFPVALSCHCGPCQIKTTDCGVFRDQPLACAPQASSSSKDPPSQPLTSTS
TPTPGASRRSSHPLPIKTSGGGGSGGGGSGGGGSKTHTCPPCPAPELLGG
PSVFLEPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYT
QKSLSLSPGK
SEQ ID NO: 6:
ATGGAAACACTGCAGGGCCTGCTGCTGTGGATGCTGCTGTCTGTGGGCGG
AGTGTGGGCCAGCAGAGGACCTCTGAGGCCCCTGTGCAGACCCATCAATG
CCACACTGGCCGCCGAGAAAGAGGCCTGCCCTATCTGCATCACCTTCACC
ACCAGCATCTGCGCCGGCTACTGCCCTTCTATGGTGCGCGTGATGCCTGC
CGCCCTGCCTGCTATTCCTCAGCCCGTGTGCACCTACAGAGAGCTGAGAT
TCGCCAGCATCAGGCTGCCCGGATGTCCTCCTGGCGTGGACCCCATGGTG
TCTTTCCCTGTGGCCCTGTCTTGCCACTGCGGCCCCTGCCAGATCAAGAC
CACCGACTGTGGCGTGTTCAGGGACCAGCCTCTGGCCTGCGCTCCACAAG
CCAGCAGCAGCTCTAAGGACCCCCCTAGCCAGCCCCTGACCAGCACCTCT
ACACCTACACCTGGCGCCTCCAGAAGAAGCAGCCACCCCCTGCCCATCAA
AACCTCTGCGGCGGAGGATCTGGGGGAGGCGGAAGCGGAGGGGGCGGATC
TAAGACCCACACCTGTCCTCCATGCCCTGCCCCTGAACTGCTGGGCGGAC
CTAGCGTGTTCCTGTTCCCCCCAAAGCCCAAGGACACCCTGATGATCAGC
AGGACCCCCGAAGTGACCTGCGTGGTGGTGGATGTGTCTCACGAGGACCC
TGAAGTGAAGTTCAATTGGTACGTGGACGGCGTGGAAGTGCACAACGCCA
AGACCAAGCCCAGAGAGGAACAGTACAACAGCACCTACCGCGTGGTGTCC
GTGCTGACCGTGCTGCACCAGGATTGGCTGAACGGCAAAGAGTACAAGTG
CAAGGTGTCCAACAAGGCCCTGCCCGCTCCCATCGAAAAGACCATCAGCA
AGGCCAAGGGCCAGCCCAGGGAACCCCAGGTGTACACACTGCCCCCCAGC
AGGGATGAGCTGACCAAGAACCAGGTGTCCCTGACCTGTCTCGTGAAGGG
CTTCTACCCCTCCGATATCGCCGTGGAATGGGAGAGCAACGGCCAGCCCG
AGAACAACTACAAGACCACACCCCCCGTGCTGGACAGCGACGGCTCATTC
TTCCTGTACAGCAAGCTGACAGTGGACAAGAGCAGATGGCAGCAGGGCAA
CGTGTTCAGCTGCAGCGTGATGCACGAAGCCCTGCACAACCACTACACCC
AGAAGTCCCTGAGCCTGAGCCCCGGCAAATAA

### Example 4

### Generation of Transgenic Mice that Produce eCG-Fc Fusion Proteins

Constructs were generated in which both of the eCG subunits were C-terminally fused to Fc portion of human IgG1 sequence (FIG. 1A-1D). The mice producing eCG-Fc fusion proteins were generated using traditional microinjection techniques. A synthetic DNA was made encoding the α subunit fused to the human IgG1 Fc sequence and the β subunit fused to the human IgG1 Fc sequence. The fused sequences were ligated into an expression vector. In these plasmids, the nucleic acid sequence encoding eCG subunit-Fc fusion proteins is under control of a promoter facilitating the expression of eCG subunit-Fc fusion protein in the mammary glands of the mice. The prokaryotic sequences were removed and the DNA microinjected into pre-implantation embryos of the mice. These embryos were then transferred to pseudo pregnant females. The progeny that resulted were screened for the presence of both eCG α-Fc and eCG β-Fc fusion transgenes. Fourteen mice were identified as carrying both fusion proteins; two mice were identified as carrying only the eCG α-Fc fusion transgene and three mice were identified as carrying only the eCG β-Fc fusion transgene.

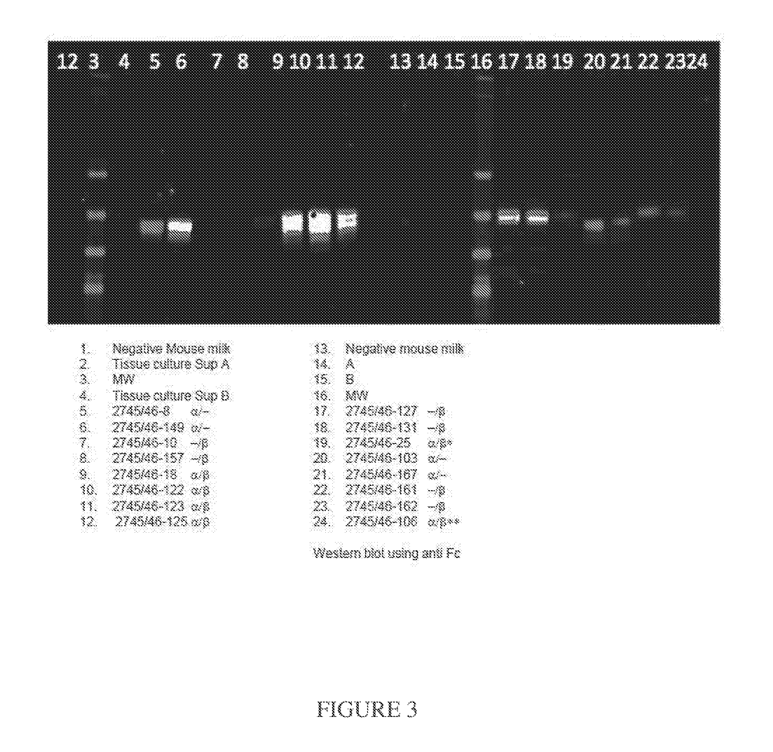

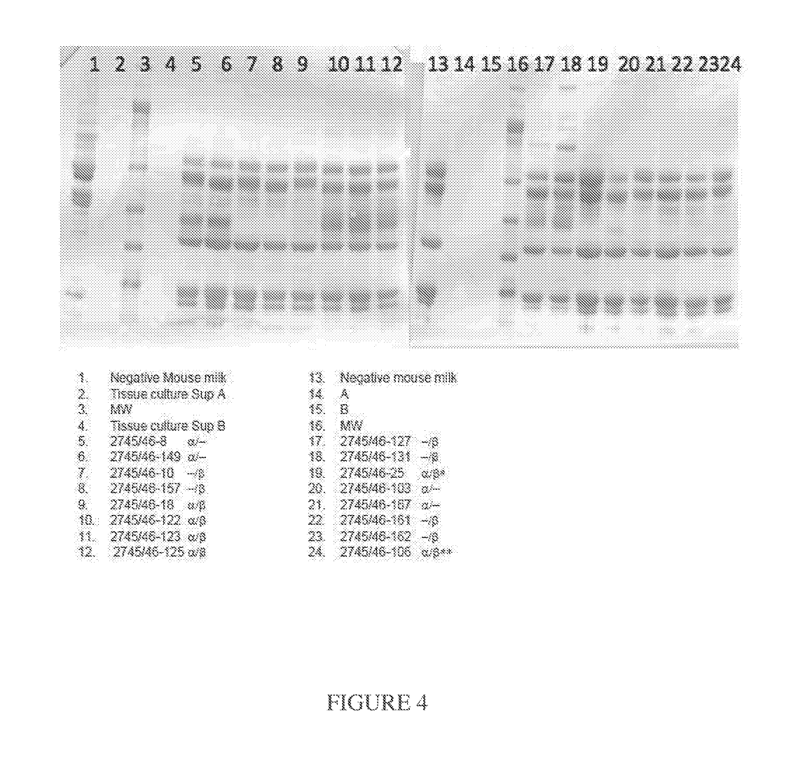

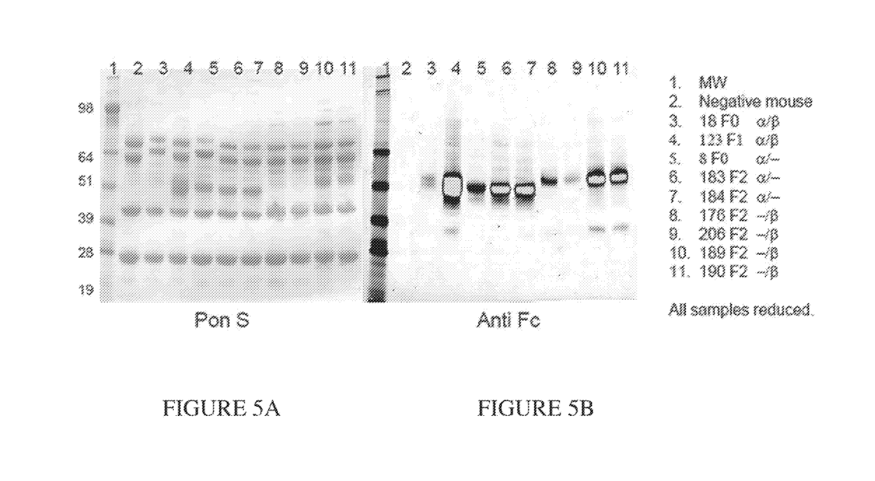

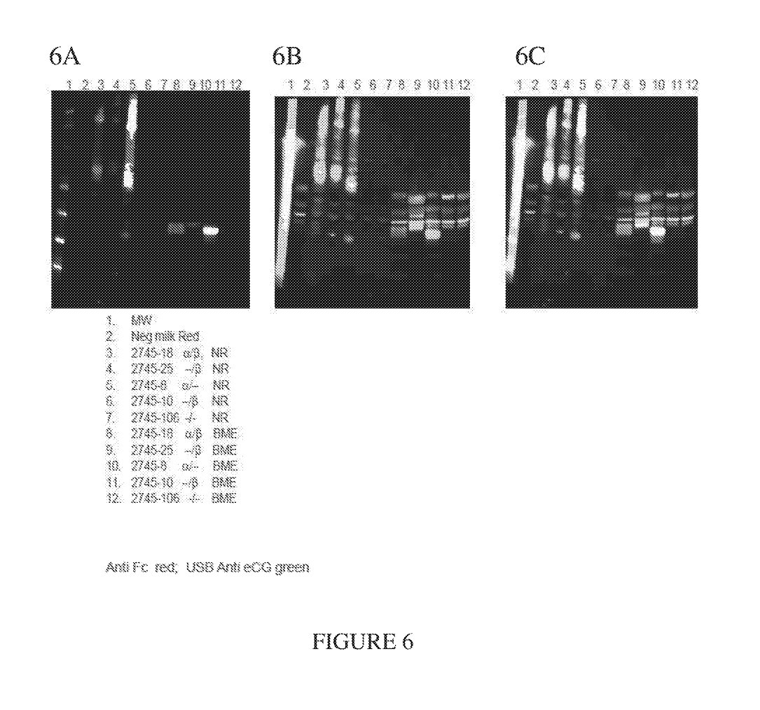

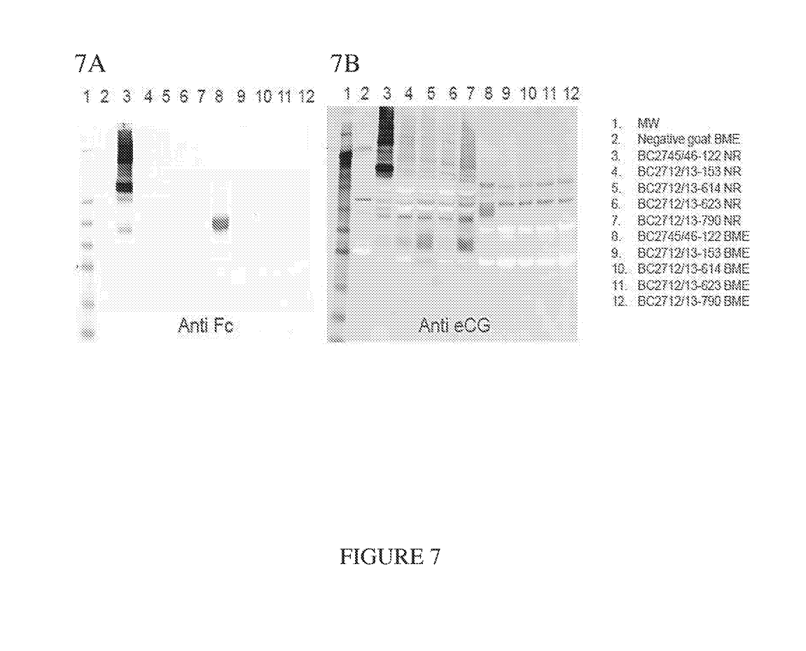

When age appropriate, the founder animals were bred to produce F1 progeny. Following pregnancy and parturition, the mice were milked. Production of eCG-Fc fusion in the milk of the transgenic mice was analyzed by Western blot using an anti-Fc primary antibody and Ponceau S protein staining, as shown in FIGS. 2-6. Milk from the transgenic mice was also analyzed by Western blot using an anti-Fc primary antibody and an anti-eCG primary antibody, as shown in FIG. 7. FIG. 7 reveals that eCG fused to Fc exhibited increased stability in the milk of transgenic mice relative to eCG that was not fused to Fc.

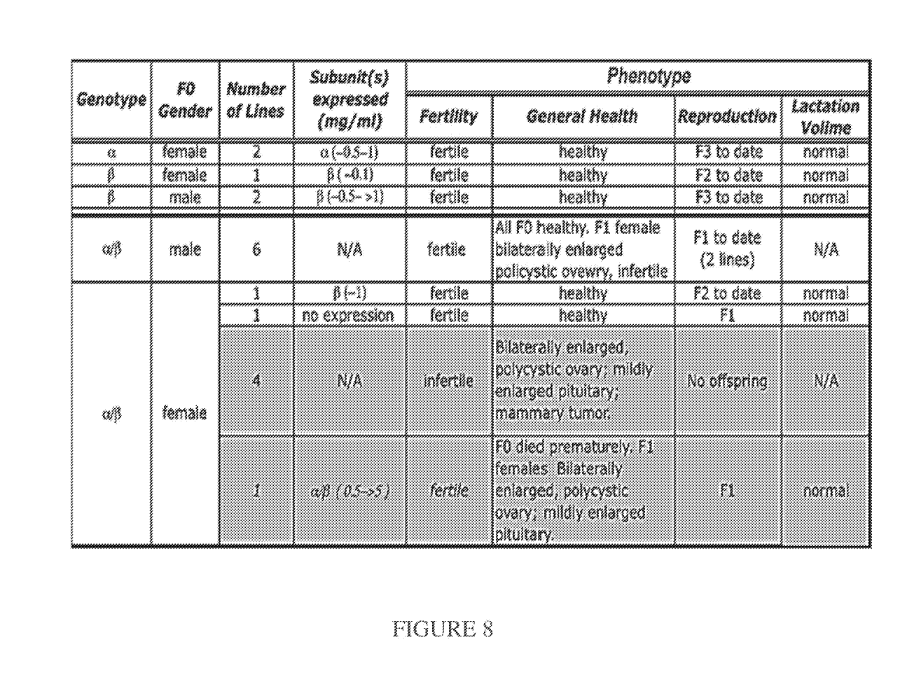

Distinct phenotypes were observed in the mice depending on the genetic background and expression level of the transgenes, as shown in Table 4 and FIG. 8.

TABLE 4
Phenotypes of eCG-Fc producing transgenic mice

| Genotype | Gender | Number of | Expression | Fertility |
|---|---|---|---|---|
| α/— | female | 2 | α/— | fertile |
| —/β | female | 1 | —/β | fertile |
| —/β | male | 1 | —/β | fertile |
| α/β | male | 7 | N/A | fertile |
| α/β | female | 1 | —/β | fertile |
| | | 1 | no expression | fertile |
| | | 4 | N/A | infertile |
| | | 1 | α/β | fertile |

### Example 5

### eCG-Fc Expression in Transgenic Goats

Transgenic goats are generated that express nucleic acid sequences encoding one or more eCG subunits fused to the human IgG1 Fc sequence (FIG. 1A-1D). The goats producing one or both eCG subunit-Fc fusion proteins are generated using traditional microinjection techniques. The fused sequences are ligated into an expression vector. In these plasmids, the nucleic acid sequence encoding eCG subunit-Fc fusion protein is under control of a promoter facilitating the expression of eCG subunit-Fc fusion protein in the mammary glands of the goats. The prokaryotic sequences are removed and the DNA microinjected into pre-implantation embryos of the goats. These embryos are then transferred to pseudo pregnant females. The progeny that result are screened for the presence of one or both of the eCG subunit-Fc transgenes.

When age appropriate, the founder animals are bred. Following pregnancy and parturition, the goats are milked. Production of eCG-Fc fusion proteins is analyzed, and the eCG-Fc fusion proteins are purified from the milk of the transgenic animals.

### Equivalents

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

What is claimed is:

1. A method comprising
providing a transgenic non-human mammal that has been modified to express a fusion protein comprising one or more polypeptide fused to an Fc domain in the mammary gland, and
harvesting the fusion protein from milk produced by the mammary gland of the transgenic non-human mammal.

2. The method of claim 1, wherein the Fc domain is a human IgG1 Fc domain.

3. The method of claim 1 or 2, wherein the sequence of the Fc domain comprises SEQ ID NO:1.

4. The method of any one of claims 1-3, wherein the fusion protein comprises more than one subunit and wherein the subunits are produced in the same transgenic non-human mammal.

5. The method of any of one of claims 1-3, wherein the fusion protein comprises more than one subunit and wherein the subunits are produced in different transgenic non-human mammals.

6. The method of claim 5, wherein the subunits are combined after being produced in different transgenic non-human mammals.

7. The method of any one of claims 1-6, wherein the transgenic non-human mammal is a bovine, porcine, caprine, ovine or rodent.

8. The method of claim 7, wherein the transgenic non-human mammal is a goat.

9. The method of claim 7, wherein the transgenic non-human mammal is a rabbit.

10. The method of any one of claims 1-9, wherein the transgenic non-human mammal has been engineered to recombinantly express a sialyltransferase, such that the fusion protein produced in said mammal has increased sialylation compared to the fusion protein produced in a transgenic non-human mammal that does not express a sialyltransferase.

11. The method of any of claims 1-10, wherein the fusion protein includes a linker region between the polypeptide and the Fc domain.

12. A composition comprising a fusion protein comprising one or more polypeptide fused to an Fc domain and further comprising milk.

13. The composition of claim 12, further comprising a pharmaceutically acceptable carrier.

14. A transgenic non-human mammal that has been modified to express a fusion protein comprising one or more polypeptide fused to an Fc domain.

15. The transgenic non-human mammal of claim 14, wherein the transgenic non-human mammal has been modified to express a sialyltransferase.

16. The transgenic non-human mammal of claim 14 or 15, wherein the transgenic non-human mammal is a bovine, porcine, caprine, ovine or rodent.

17. The transgenic non-human mammal of claim 16, wherein the transgenic non-human mammal is a goat.

18. The transgenic non-human mammal of claim 16, wherein the transgenic non-human mammal is a rabbit.

19. A method comprising
administering an effective amount of a transgenically produced fusion protein comprising one or more polypeptide fused to an Fc domain to a subject.

20. The method of claim 19, wherein the subject is a human or non-human mammal.
